(19) United States
(12) Patent Application Publication
(10) Pub. No.: US 2014/0187904 A1
(43) Pub. Date: Jul. 3, 2014

(54) METHOD AND SYSTEM FOR DETERMINING WHETHER ARTERIAL TISSUE COMPRISES ATHEROSCLEROTIC PLAQUE

(71) Applicants: Marjan RAZANI, Toronto (CA); Adrian Linus Dinesh MARIAMPILLAI, Toronto (CA); Victor X. D. YANG, Toronto (CA); Michael KOLIOS, Ancaster (CA)

(72) Inventors: Marjan RAZANI, Toronto (CA); Adrian Linus Dinesh MARIAMPILLAI, Toronto (CA); Victor X. D. YANG, Toronto (CA); Michael KOLIOS, Ancaster (CA)

(21) Appl. No.: 14/141,584

(22) Filed: Dec. 27, 2013

Related U.S. Application Data
(60) Provisional application No. 61/746,642, filed on Dec. 28, 2012.

(51) Int. Cl. A61B 5/00

(52) U.S. Cl. CPC A61B 5/0097
USPC 600/407

(57) ABSTRACT

Provided herein are methods and systems to determine at least one mechanical property of a tissue sample and, based upon the determined at least one mechanical property, whether the tissue sample comprises atherosclerotic plaque. The method comprises generating a shear wave in an arterial tissue sample by applying an acoustic impulse thereto; measuring propagation of the shear wave via an optical coherence elastography apparatus; determining at least one mechanical property of the arterial tissue sample based on the propagation of the shear wave; and comparing the at least one mechanical property of the arterial tissue sample to a reference data set to determine whether the arterial tissue sample comprises atherosclerotic plaque. A hybrid optical coherence tomography imaging/acoustic radiation force impulse system is disclosed.

### FIELD

The specification relates Optical Coherence Elastography (OCE), and specifically to a method and system for determining whether arterial tissue comprises atherosclerotic plaque.

### BACKGROUND

Atherosclerosis is a disease of arteries which is associated with lipid deposition and plaque formation. The various components of atherosclerotic plaques may be predictors of different kind of outcomes of the disease. Thus, it is of interest to identify and characterize the atherosclerotic plaque components.

### SUMMARY

According to a non-limiting implementation, there is provided a method comprising: generating a shear wave in an arterial tissue sample by applying an acoustic impulse thereto; measuring propagation of the shear wave via an optical coherence elastography apparatus; determining at least one mechanical property of the arterial tissue sample based on the propagation of the shear wave; and comparing the at least one mechanical property of the arterial tissue sample to a reference data set to determine whether the arterial tissue sample comprises atherosclerotic plaque.

According to an aspect of the non-limiting implementation, the shear wave is generated using an ultrasound transducer.

According to an aspect of the non-limiting implementation, the acoustic impulse comprises an acoustic radiation impulse force.

According to an aspect of the non-limiting implementation, the optical coherence elastography apparatus comprises an acoustic radiation force-optical coherence elastography.

According to an aspect of the non-limiting implementation, the optical elastography apparatus comprises a swept source optical tomography system.

According to an aspect of the non-limiting implementation, the at least one mechanical property comprises one or more of a Young's modulus and a shear modulus.

According to another non-limiting implementation, there is provided a system comprising: a transducer for generating a shear wave in an arterial tissue sample by applying an acoustic impulse thereto; an optical coherence elastography apparatus for measuring propagation of the shear wave via; and a computer enabled to determine at least one mechanical property of the arterial tissue sample based on the propagation of the shear wave, and compare the at least one mechanical property of the arterial tissue sample to a reference data set to determine whether the arterial tissue sample comprises atherosclerotic plaque.

### BRIEF DESCRIPTIONS OF THE DRAWINGS

For a better understanding of the various implementations described herein and to show more clearly how they may be carried into effect, reference will now be made, by way of example only, to the accompanying drawings in which:

FIG. 1 depicts a shear wave generated in a tissue sample, including an enlarged view of the focal point of the generated shear wave (right-hand side image), according to non-limiting implementations.

FIG. 2 depicts a system utilizing acoustic radiation force-optical coherence elastography (ARF-OCE) that can be used to determine at least one mechanical property of a tissue sample, according to non-limiting implementations.

FIGS. 3(a) to (f) depict optical coherence tomography (OCT) images of a titanium dioxide-gelatin phantom taken by a swept source OCT (SS-OCT) system, according to non-limiting implementations.

FIG. 3(g) depicts an isophase curve based upon results of a shear wave generated according to non-limiting implementations.

FIG. 4 depicts a flowchart of a method used to determine whether an arterial tissue sample comprises atherosclerotic plaque, according to non-limiting implementations.

FIG. 5 depicts an acoustic radiation force-optical coherence elastography (ARF-OCE) system that can be used to detect shear waves in vivo and to help determine whether an arterial tissue sample comprises atherosclerotic plaque, according to non-limiting implementations.

### DETAILED DESCRIPTION

Elastography is a method of generating stiffness and strain images of soft tissues for diagnostic purposes. An imaging modality can be used to detect tissue deformation behaviors under static or dynamic load and present the resulting images as elastograms. Elastograms contain information about local variations of stiffness inside a region of interest, as well as additional clinical information such as the identification of suspicious lesions, the diagnosis of various disease states, and the monitoring of the effectiveness of treatments.

The elastic properties of tissues are related to the underlying structure of the tissue and are strongly affected by pathological changes. For example, edema, fibrosis, and calcification alter the elastic modulus of the extracellular tissue matrix. Different tissues within atherosclerotic plaque can have distinctive elastic properties and cancerous tumors are often stiffer than benign and normal tissue. Elastography has been used to assess breast or brain tissue for malignancy and atherosclerotic arteries for vulnerability to myocardial infarction.

Different imaging modalities, such as ultrasound (US) imaging or magnetic resonance imaging (MRI), can be used to measure tissue displacements and estimate the resulting tissue mechanical properties. The disadvantages of MRI include cost, long clinical wait times, and technological complexity. As well, both US and MRI have spatial resolutions in the order of 0.1-1 mm, which is usually insufficient for detecting small and subtle elastic variations in tissues, such as in small tumors and atherosclerotic plaques.

Optical coherence tomography (OCT) is an optical tomographic imaging technique that shares many similarities to US imaging despite using light. OCT may have several advantages over other imaging modalities, primarily due to its inherently high resolution, which allows for the identification of micron sized morphological tissue structures. Furthermore, OCT equipment is usually inexpensive and its interchangeable components can enable experiment-specific flexibility.

Optical coherence elastography (OCE) is a relatively new elastography technology that uses OCT to measure tissue displacement and biomechanical properties of soft tissues. During OCE, tissues can be excited internally or externally, as well as statically or dynamically. Methods for creating dynamic compressions include acoustic radiation force (ARF) and low-frequency vibrations with a needle.

ARF excitation for producing transient excitations has been implemented to assess the mechanical properties of tissues. ARF imaging is used in general elasticity imaging methods, for the characterization of lesions, muscle screening, and imaging of the calcification of arteries.

ARF has also been used for the internal mechanical excitation of a sphere embedded in a gelatin phantom, with phantom deformations detected with a spectral domain OCT (SD-OCT) system and recorded as M-mode phase images and then the displacement of the sphere over time was used for shear modulus measurements. Tissue velocity and strain measurements have been obtained via tissue imaged under mechanical loading with a vascular OCE protocol specific to the exploring of tissue biomechanics. Furthermore, strain responses of a tissue phantom undergoing compressive forces have been measured using speckle tracking and SD-OCT methods for detecting small and large deformations. Spectroscopic OCE (S-OCE) has been utilized for frequency-dependent contrast of the displacement amplitude and phase of a silicone phantom, with ex vivo tumor follow-up imaging, in B-mode OCT imaging with applications in pathology.

As well, a dynamic SD-OCE technique has been applied to three-layer silicone tissue phantoms and ex vivo rat tumor tissue has been reported to provide contrast between sample regions with different mechanical properties, thus to mechanically characterize tissue.

In vivo three-dimensional OCE has also been implemented to observe elastic properties of superficial skin, which can be utilized for detecting strain rates and contrast useful for pathologists. A ring actuator has been applied to in vivo dynamic OCE to enable excitation and imaging for the same side of the sample, thus providing an alternative for contrast in OCT images.

Methods and systems for determining mechanical properties of arterial tissues samples by propagating shear waves in arterial tissue samples with ARF, and measuring the shear wave speed and its associated properties with OCT phase maps are provided herein using an ARF/OCE system. The OCT phase maps are acquired with a swept-source OCT (SS-OCT) system. According to some implementations, the phase noise of a relatively low speed SS-OCT (e.g. 8 kHz bi-directional) is sufficiently low to measure phase changes induced by shear wave propagation.

The described dynamic excitation OCE methods and systems use ARF as the excitation source.

The speed of the generated shear wave can be measured in several ways. For example, by applying the inversion of the Helmholtz Equation, which characterizes the shear wave propagation, using algorithms that measure lateral time for peak-to-peak displacements, tracking the displacement field jitters that are associated with shear waves, and using a variety of correlation-based algorithms. The speed of shear waves that propagate in soft tissues is directly related to the shear modulus of the material.

Traditional compression wave imaging methodologies, such as US, provide measurements based on the tissue bulk modulus, which is confined to a relatively small range for soft tissues. However, the shear modulus for soft biological tissues actually span a much larger range compared to the bulk modulus by several orders of magnitude.

The shear modulus can be used as a cancer biomarker as determined using ultrasonic techniques. Prior to discussing arterial samples, however, a successful prototype for performing shear modulus measurements of homogeneous tissue equivalent phantoms from OCT phase elastograms, without the requirement of measuring displacements of embedded targets will be described. This methodology, referred to herein as Shear Wave OCE (SW-OCE), can be useful can also be used for determining mechanical properties of heterogeneous tissue structures, and can also be applied to applications in pathology, intravascular studies, US/OCT needle probe imaging, and small animal studies.

Acoustic Radiation Force (ARF):

Acoustic radiation force (ARF) is produced by a change in the energy density of the incident acoustic field. ARF is generated by the transfer of momentum from the acoustic wave to the tissue. This force can be applied in the direction of the longitudinal wave propagation and the magnitude of the force can be approximated by:

$$F = \frac{2 α I}{C} . \qquad ( 1 )$$

where F, kg/(s ² cm ²), is the acoustic radiation force, C (m/s) is the speed of sound in the medium to which the ARF is being applied, α (Np/m) is the absorption coefficient of the medium and I (W/cm²) is the temporal average intensity at a given spatial location.

According to some implementations, shear waves are generated in the medium using a focused impulse produced by an ultrasound transducer. The focused impulse creates a displacement in the direction of ultrasonic beam propagation which is largest at the transducer focus. After the impulse, the medium relaxes back to its original state producing a shear wave. The shear wave propagates in the direction perpendicular to the direction of the focused US propagation.

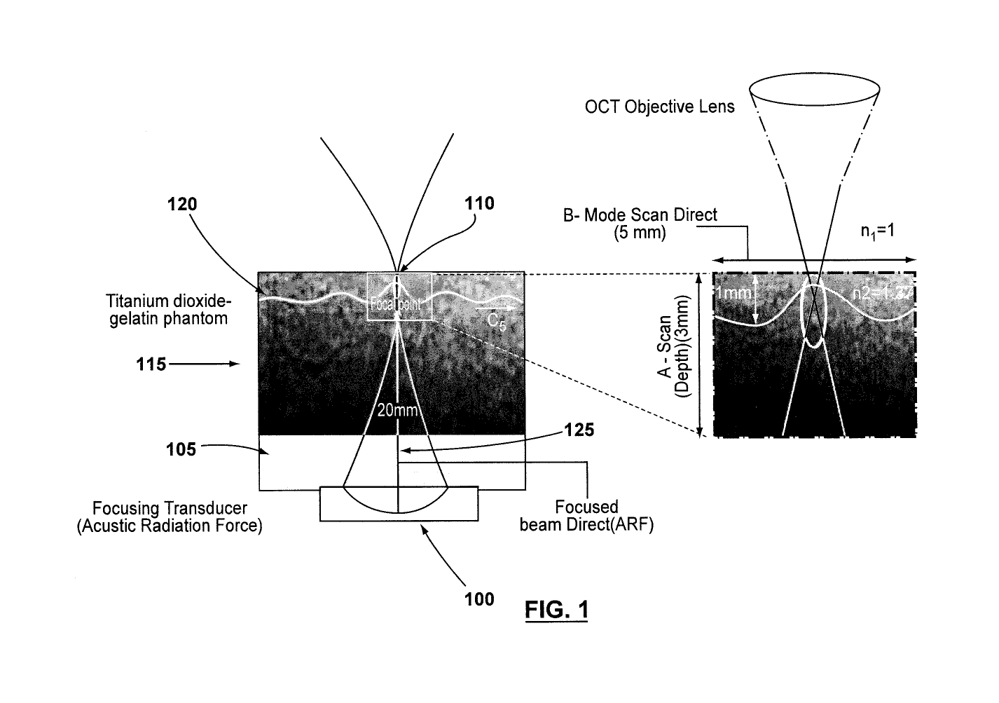

For example, as depicted in FIG. 1, which depicts a non-limiting example of generating a shearwave in a medium, a focused ultrasound transducer 100 generates a focused beam 125 through a couplant gel 105 at a focal point 110, located in a phantom 115 (which, in a non-limiting example, comprises a titanium dioxide-gelatin phantom). Shear waves, such as shear wave 120, are produced at focal point 110 and travel through phantom 115 in the direction indicated by the white arrow labeled Cₛ.

According to some implementations, focused ultrasound transducer 100 has a focal depth of about 20 mm. However other focal depths are within the scope of present implementations.

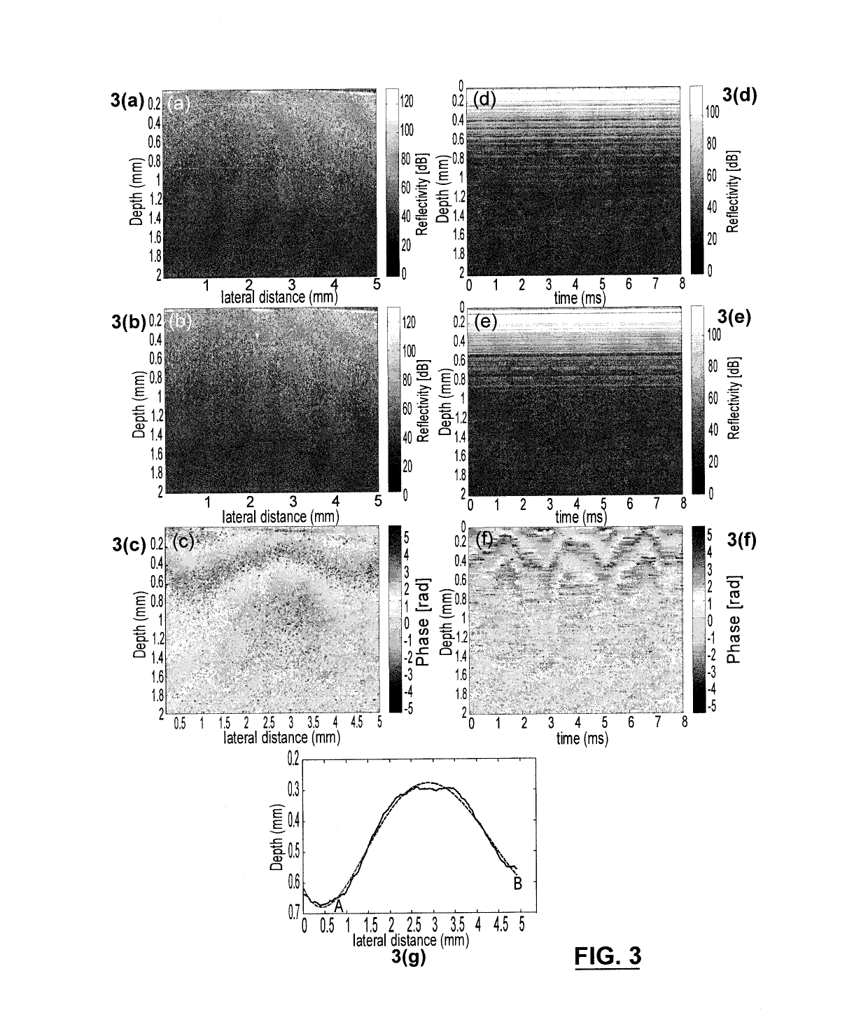

According to some implementations, B-mode OCT images, such the images depicted in FIGS. 3(a), (b), described in detail below, are taken at the focal point for phase map analysis.

By using the Voigt model for a homogenous medium, the shear wave speed Cₛ can be related to the shear modulus μ₁ of the medium, shear viscosity μ₂ of the medium, shear wave angular frequency ω of the medium, and tissue density ρ of the medium, as follows:

C_{S}(ω)=\sqrt{2(μ₁²+ω²∥₂²)/ρ(μ₁+\sqrt{μ₁²+ω²μ₂²})} (2)

According to some implementations, the displacements of shear waves at each tracking location are calculated with a speckle-tracking algorithm based on the OCT phase maps generated. The shear wave speed Cₛ can be calculated using Δφ and Δr measurements obtained from the measured phase shift, and the distance between the two tracking locations, respectively. The shear wave speed Cₛ can be calculated using the following equation:

$${C}_{S} ( ω ) = \frac{ω Δ r}{Δ ϕ} . \qquad ( 3 )$$

where ω=2πf, Δφ is the phase shift between two tracked locations, and Δr is the distance between the two tracked locations. The shear wave frequency (f) is dependent on several factors, such as the beam width of the excitation transducer.

The Young's modulus and shear modulus are helpful in defining the mechanical properties of the medium. If an isotropic homogenous medium (i.e. phantom medium) is assumed, the Young's modulus and shear modulus can be calculated using the following equation:

$$\begin{matrix}{C}_{s} = \sqrt{\frac{μ}{ρ}} . \\ and & ( 4 ) \\ E = 2 ( 1 + v ) μ \approx 3 μ = 3 {C}_{S}^{2} ρ . & ( 5 )\end{matrix}$$

where μ is the shear modulus, ρ is the density, and v is Poisson's ratio. For example, assuming that soft tissues are close to incompressible, with a constant Poisson's ratio of about 0.5.

Example Implementation:

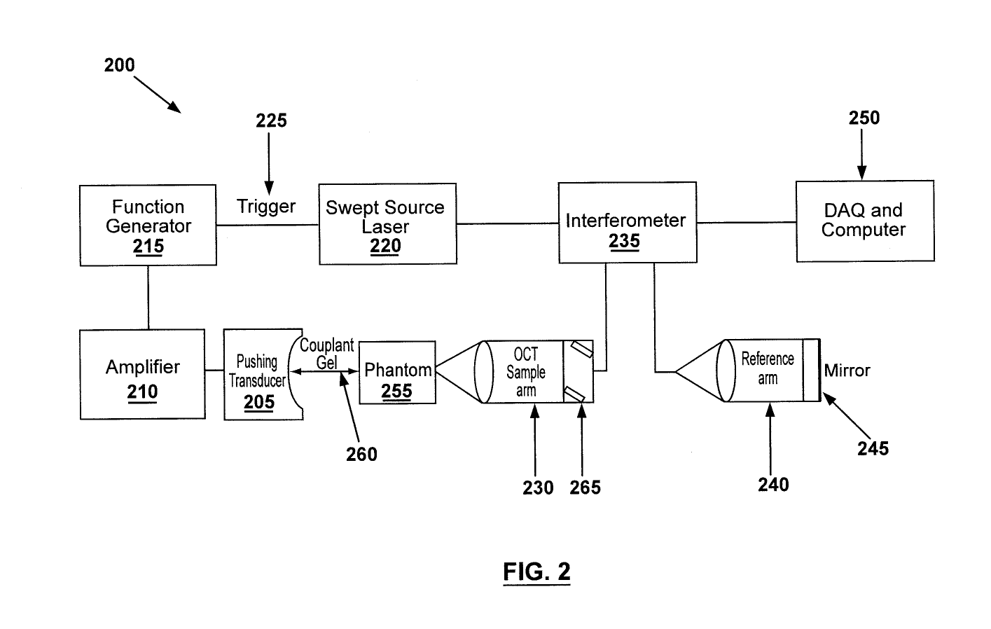

Attention is directed to FIG. 2, which depicts system 200 for determining whether arterial tissue comprises atherosclerotic plaque. System 200, utilizes acoustic radiation force-optical coherence elastography (ARF-OCE) to determine at least one mechanical property of a tissue sample, according to non-limiting implementations. System 200 comprises ultrasound pushing transducer 205, amplifier 210 to amplify the voltage provided to pushing transducer 205, function generator 215 enabled to excite pushing transducer 205, swept source laser 220, trigger 225 for activating swept source laser 220, OCT sample arm 230 comprising sample arm mirror 265, interferometer 235, reference arm 240, reference mirror 245, data acquisition module (DAQ) and computer 250, and a beam splitter (not shown).

According to some implementations, function generator 215 comprises an Agilent 33250A 80 MHz Function/Arbitrary Waveform Generator that is synchronized with the swept source laser 220, pushing transducer 205, amplifier 210 and interferometer 235 of system 200. According to some implementations, function generator 215 is enabled to excite pushing transducer 205 at a frequency of about 20 MHz.

According to some implementations, pushing transducer 205 comprises a focused ultrasound transducer.

Couplant gel 260 is used as a medium to transfer the ultrasound waves generated by pushing transducer 205 to phantom 255, used to model a tissue sample. According to some implementations, phantom 255 comprises a titanium dioxide-gelatin phantom.

Provided below are descriptions of example implementations of the described methods utilizing ARF-OCE as a dynamic excitation OCE technique using ultrasound ARF as the excitation source, as reduced to practice. These examples were implemented using swept source OCT (SS-OCT) system 200, also referred herein as system 200. These examples are provided for illustrative purposes and to facilitate understanding of the described methods and systems utilizing ARF-OCE. It is understood that these examples are not specifically limiting variations thereof and are also within the scope of the claimed implementations.

According to one example implementation, swept source laser 220 had a center wavelength of about 1310 nm, a bandwidth of about 110 nm, and an A-scan rate of about 8 kHz. The lateral resolution was about 13 μm in the tissue samples. The ARF (i.e. internal mechanical excitation) was applied using pushing transducer 205 which, in non-limiting example implementations comprises a circular piezoelectric transducer element operating at about 20 MHz and having a diameter of about 8.5 mm, an f-number of about 2.35, and transmitting sine-wave bursts of about 400 μs. Pushing transducer 205 was excited by function generator 215. According to some implementations, pushing transducer 205 comprises lead zircon titanate.

OCT images were taken with a swept-source system comprising swept-source 220, OCT sample arm 230, interferometer 235, reference arm 240, reference mirror 245, and DAQ and computer 250. B-mode images were taken of phantom 255, which in a non-limiting example comprises a titanium dioxide-gelatin phantom of about 5 mm in length. The OCT image A-scan depth was about 3 mm. The focal point of pushing transducer 205 used for the ARF experiments was located about 20 mm from pushing transducer surface 265 and about 1 mm below the top surface of phantom 255.

M-mode images similar to FIG. 1, were taken along the y-direction (i.e. parallel to the ARF beam), the direction of the ARF beam. Shear waves typically propagate radially outwards from the focal point, perpendicular to the direction of the ARF beam (i.e. the shear waves propagate along the x-axis). The pushing transducer 205 push sequence was synchronized with the OCT imaging triggering system, comprising swept-source laser 220, OCT sample arm 230, interferometer 235, reference arm 240, reference mirror 245, and DAQ and computer 250. The US depth of field was about 2.94 mm, with US focus of about 20 mm. The full width at half maximum was calculated to be about 246 μm.

In non-limiting example implementations, phantom 255 comprised gelatin mixed with titanium dioxide, which provided uniform acoustic and optical scattering. To prepare phantom 255, gelatin powder (Type B, Fisher Scientific, G7-500) and distilled water were heated in a water bath at about 60-65° C. for one hour and periodically stirred. Two tissue phantoms 255 with different gelatin concentrations were prepared (Phantom 1: about 14%, Phantom 2: about 8%). When the phantom samples cooled to about 45° C., about 0.1% weight by weight titanium dioxide (Sigma-Aldrich, Titanium (IV) oxide nanopowder, less than about 25 nm particle size, about 99.7% trace metals basis) was added and thoroughly mixed. The phantom solution was poured into rectangle molds (about 20 mm height) and allowed to congeal. The titanium dioxide was used as a scattering agent.

The OCT signals from phantoms 255 were used for the measurement of the shear wave speed and mechanical properties. Pushing transducer 205 was synchronized with the OCT imaging system. The phase analysis was applied to B-mode and M-mode OCT images, which were obtained while pushing transducer 205 was generating the “push” (i.e. the ARF excitation/induced displacement) in each phantom. A fast Fourier transform was performed on the OCT data, and phase maps of phantom 255 under US loading were generated, which are related to the ARF induced displacement in the phantom.

In order to create a reference data set, independent measurements of the mechanical properties of the phantom were made using a rheometer (not shown). According to some implementations, the reference data set can comprise one or more reference data points. Material properties of the same gelatin gels (having about 14% w/w and about 8% gelatin concentrations) were imaged using SW-OCE were tested in a parallel-plate shear rheometer in oscillatory mode, and specifically using a Physica MCR 301 rheometer (Anton Paar GmbH, Graz, Austria) equipped with a Peltier plate temperature control unit (P-PTD 200). The parallel plate measuring geometry (PP 25/TG) with a diameter of about 25 mm was used. The frequency dependent elastic and viscous moduli, G′ and G″, were measured for samples aged for about 1 hour at about 25° C. for frequencies ranging from about 10 Hz to about 10² Hz. To avoid sample drying, the measuring geometry was covered with a solvent trap containing a moist strip of paper tissue. Preceding each measurement, the temperature of the Peltier plate was set at about 10° C. and the mixed hot biopolymer solution was poured directly onto the cold plate. The quenched sample transformed to a gel, after which the temperature of the rheometer cell was raised from about 10° C. to about 25° C. at a rate of about 6° C. min⁻¹. The upper cone was then lowered onto the sample to an operating gap width (about 1 mm) and the sample was trimmed and held at about 25° C. for about 10 minutes. Using this thermal treatment, a conventional gel state condition was satisfied for all samples. After thermal treatment, rheological measurements at about 25° C. were performed.

To obtain the moduli (e.g. Young's modulus and shear modulus) at the dominant frequency of the shear waves generated in the SW-OCE experiments, the rheometer shear modulus versus frequency data was extrapolated to a value of interest.

OCT images of phantom 255 were taken with system 200 and are shown in FIGS. 3(a) to (f). B-mode and M-mode images of phantom 255, as well as their respective phase maps provide information that is required to calculate distance, the phase shift between two locations and the shear wave frequency.

FIGS. 3(a), (b) depict B-mode OCT structural images and FIG. 3(c) depicts the corresponding B-mode phase map of phantom 255 (titanium dioxide-gelatin phantom (14%)). Box 305 (dashed lines) in FIG. 3(a) represents the location of the superimposed fitted sine wave observed in the phase map. Arrow 310 in FIG. 3(b) indicates the position where the M-mode OCT images (FIGS. 3(d), (e)), with the ARF on and off, respectively, were acquired and synchronized with the OCT swept-source wavelength sweep.

The corresponding B-mode phase map of phantom 255 was used to measure Δr and Δφ or the calculation of the shear wave speed. The scale depicted in FIGS. 3(c), (f) represented the change of the phase value (radians). The M-mode phase map (FIG. 3(f)) from phantom 255 was used to calculate the shear wave frequency. To better illustrate the calculation of Δr, MATLAB was used to plot an isophase curve which now shows the experimental data (jagged curve). The smooth curve is a best fit with a polynomial (FIG. 3(g)).

By measuring the time difference between successive troughs in the M-mode dataset of FIG. 3(f), the dominant frequency of the shear wave was calculated to be about 266 Hz (for both phantoms 255). The shear wave group speed was then calculated by using the Δr and Δφ obtained from FIG. 3(c, g), which depicts the distance between the two successive locations and the measured phase shift, respectively. Two successive locations can be chosen at a particular depth z. In this example implementation, z was chosen to be about 0.6 mm. At these two locations in the image, phase values are retrieved. The calculation of Δφ involves the measurements of the two phase values at the aforementioned two locations, which are then used in equation (3) to calculate the shear wave group velocity.

Another way to illustrate a “snapshot” of the shear wave is to plot an isophase curve. In this example implementation, an isophase curve was generated by averaging the phase value between depths from about 0.25 mm to about 0.7 mm FIG. 3(g). These values were then used in equation (3) to calculate the shear wave speed. The calculated shear wave speeds for the 14% and 8% gelatin-titanium dioxide phantoms were 2.24±0.06 m/s and 1.490.05 m/s, respectively, and reported in Table A (shown below). The average values and standard deviations were calculated from 10 different pairs of locations in the phase maps for all calculations of Δr and Δφ.

The mechanical properties of Young's modulus and shear modulus were also calculated using the above results. The measured density p of the phantom samples was about 1050 kg/m³. Table A summarizes the Young's moduli and shear moduli for both phantoms. The shear modulus estimated using SW-OCE for Phantom 1 (14%) was about 5.3±0.2 kPa and for Phantom 2 (8%) was about 2.3±0.1 kPa. The errors for the SW-OCE results represent the standard deviation. As expected, the values of the Young's moduli and shear moduli were greater for the phantom with the higher concentration of gelatin. The shear moduli of both phantoms calculated using SW-OCE was compared to the shear moduli of the same two phantoms measured by the rheometer in Table. A. The errors for the rheometer results represent the standard deviations.

TABLE (A)
The mechanical properties of the phantoms.

| | Shear wave | | |
|---|---|---|---|
| | speed | Shear modulus | Young modulus |
| Samples | (Cₛ, m/s) | (μ, kPa) | (E, kPa) |
| Phantom 1 | 2.24 ± 0.06 | 5.3 ± 0.2 | 15.8 ± 0.6 |
| (14%) | | | |
| Rheometer | | 4.93 ± 0.05 | |
| Phantom 2 | 1.49 ± 0.05 | 2.3 ± 0.1 | 7.0 ± 0.3 |
| (8%) | | | |
| Rheometer | | 2.06 ± 0.09 | |

In summary, described above are methods and systems that employ a SW-OCE technique using ARF for mechanical excitation of a homogeneous gelatin phantom to measure shear wave propagation. The mechanical excitation produces motions within the phantom that can be used for the estimation of mechanical properties using SW-OCE. This excitation produces shear waves that propagate perpendicular to the US beam. The close proximity of the transducer focus to the surface of the phantom suggests that the surface (i.e. Rayleigh) waves were produced. A discrepancy between the values provided by the rheometer and the SW-OCE technique can be related to the extrapolation required from the rheometer data to obtain the values of the shear modulus at about 266 Hz and as the SW-OCE technique, as implemented, can be more sensitive to the shear wave group velocity, whereas the shear modulus from the rheometer is reported at one frequency. These SW-OCE techniques can also be used with in vivo clinical applications including pathology, intravascular studies, US/OCT catheter imaging, and small animal studies due to the potential for measuring mechanical properties within tissues for making disease assessments. More specifically, these techniques can be used to determined whether arterial samples comprise atherosclerotic plaque, as will be presently described.

Example Implementation: Detecting Atherosclerotic Plaque

A further application of the above-described techniques is to determine whether an arterial tissue sample comprises atherosclerotic plaque. For example, intravascular OCE may be used for detecting rupture-prone (i.e. vulnerable) plaque.

The main components of atherosclerotic plaque include a large extracellular necrotic core with a thin fibrous cap infiltrated by macrophages, all of which have varying biomechanical properties. Rupture of the cap induces the formation of a thrombus, which can obstruct the coronary artery, causing an acute heart attack and frequently results in patient death. Detailed characterization of the arterial wall biomechanics can provide complementary information to quantify lesion stability. Both in vitro and in vivo studies have revealed that strain is higher in fatty arterial tissue components than in fibrous plaques. The presence of a high-strain area that is surrounded by a low-strain region can assist in the identification of vulnerable plaque with high sensitivity and specificity. Thus, changes in the mechanical properties of the arterial tissue can be detected, quantified, and correlated with clinical symptoms and inflammatory markers.

Intravascular elastography can be used to correlate elastograms with histological characteristics of the blood vessel wall. Intravascular ultrasound (IVUS) elastography has been successfully used to extract arterial radial strains with a spatial resolution of 200 μm. For example, ultrasound elastography within carotid walls induced by the natural cardiac pulsation, and coupled to a simple inverse problem, has been used to recover the wall elastic modulus at the blood pulsatility frequency (1 Hz). Further progress has been made by adapting the dynamic microelastography method and formulating an inverse problem to study the radial viscoelasticity of the vessel wall. Although this has provided some success in plaque identification, there exist several limitations because vulnerable atherosclerotic plaques have structural components (e.g., fibrous caps) on the order of 50-200 μm, which lie below the resolvable limit of the IVUS imaging system. However, OCT, as implemented herein can overcome these hurdles due to its inherent high resolution and noninvasive near-cellular-level imaging for plaque quantification.

An application of intravascular OCT is the detection of thin-cap fibroatheroma (TCFA), an important type of vulnerable plaques. Indeed, with high spatial resolution and, thus, the ability to visualize structures on the size scale of thin fibrous caps, intravascular OCE can provide high-resolution characterization of strains in tissue lying within 1.0-1.5 mm of the lumen interface, a region susceptible to plaque disruption.

OCT can also be used as a basis for finite element analysis and results comparable to histology methods for stress and strain analysis of atherosclerosis can be obtained. OCT-based finite element analysis can be a powerful tool for investigating coronary atherosclerosis and detecting vulnerable plaque. The capability of OCE to resolve TCFA is advantageous when compared to competing technologies, such as IVUS. A large stiffness variation near the lumen is generally assumed mechanically unstable. Therefore, the creation of an arterial shear modulus and Young's modulus image through OCE can be helpful in establishing a diagnostic tool for assessment of plaque vulnerability.

Hence, described herein are methods and systems that use acoustic radiation force impulse (ARFI) ultrasound, to perform elastography. ARFI ultrasound uses high intensity acoustic impulses to remotely displace arterial tissue and generate a shear wave in a known location (i.e. a transducer focal point). Another technology is used to detect the shear wave propagation to estimate the shear wave velocity and the mechanical properties of the arterial tissue at the known location, specifically OCE, as described above. Mechanical properties such as the shear modulus and Young's modulus are measured and compared to reference values (e.g. in a reference data set) of the atherosclerosis plaques.

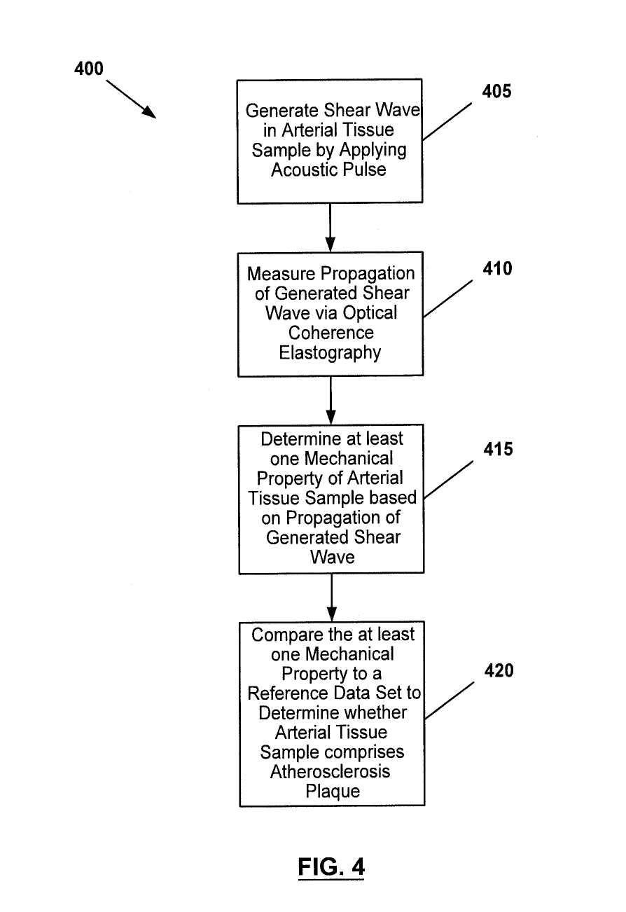

FIG. 4 depicts a flowchart of method 400 used to determine whether an arterial tissue sample comprises atherosclerotic plaque, according to non-limiting implementations. It is to be emphasized, however, that method 400 need not be performed in the exact sequence as shown, unless otherwise indicated; and likewise various blocks may be performed in parallel rather than in sequence; hence the elements of method 400 are referred to herein as “blocks” rather than “steps”.

At block 405, a shear wave in an arterial tissue sample is generated by applying an acoustic impulse to the arterial tissue sample, similar to generation of a shear wave as described above that is described with respect to phantoms. According to some implementations, the shear wave is generated by an ultrasound transducer.

At block 410, propagation of the generated shear wave is measured via an optical elastography apparatus.

At block 415, at least one mechanical property of the arterial tissue sample is determined, based on the propagation of the shear wave. For example, mechanical properties such as the Young's modulus and the shear modulus of the arterial tissue sample can be determined at block 415.

At block 420, the at least one mechanical property of the arterial tissue sample determined at block 415 is compared to a reference data set to determine whether the arterial tissue sample comprises atherosclerotic plaque. According to some implementations, the reference data set is derived from independent measurements of the at least one mechanical property using a rheometer in an arterial tissue sample that is known to comprise at least some atherosclerotic plaque.

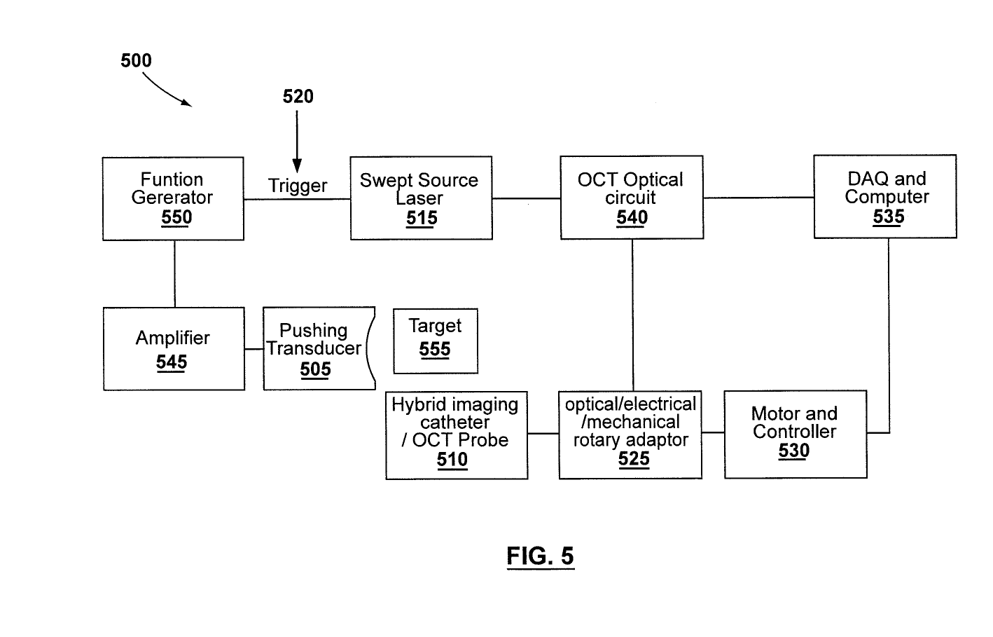

To detect shear waves in vivo, a hybrid OCT imaging/ARFI system is used. FIG. 5 depi cts acoustic radiation force-optical coherence elastography (ARF-OCE) system 500, also referred to herein as system 500, which can be used to determine the mechanical properties of an atherosclerotic plaque in vivo. The following discussion of system 500 will lead to a further understanding of method 400 and its various features. However, it is to be understood that system 500 and/or method 400 can be varied, and need not work exactly as discussed herein in conjunction with each other, and that such variations are within the scope of present implementations.

In system 500, an ultrasound transducer, such as depicted pushing transducer 505, and an OCT probe are located within a catheter, depicted as hybrid imaging catheter/OCT probe 510 (also referred to herein as hybrid (OCT imaging/ARF) catheter 510). According to some implementations, hybrid (OCT imaging/ARF) catheter 510 comprises a rotating catheter. The pushing transducer 505 produces a shear wave in arterial tissue sample 555 that propagates outwards from the focal point of pushing transducer 505 (for example, as depicted in FIG. 1) and inside to the location of the blood vessel/atherosclerotic plaque that is to be analysed.

System 500 comprises an OCT system, which can comprise a custom SS-OCT system comprising swept-source laser 515, DAQ and computer 535, and OCT optical circuit 540, and further comprises swept source laser 515 as the light source that is activated by trigger 520, a motor system that comprises motor and controller 530, OCT image processing software implemented by a computing device, such as data acquisition (DAQ) module and computer 535, a rotary joint that facilitates mechanical, electrical, and optical connections between the hybrid (OCT imaging/ARF) catheter 510 and stationary imaging hardware, such as OCT optical circuit 540, and pushing transducer 505. According to some implementations, laser 515 utilizes wavelength of about 1310 nm and a bandwidth of about 110 nm.

DAQ and computer 535 is enabled to determine at least one mechanical property of the arterial tissue sample based on the propagation of the shear wave, and compare the at least one mechanical property of the arterial tissue sample to a reference data set to determine whether the arterial tissue sample comprises atherosclerotic plaque.

Pushing transducer 505 is in communication with amplifier 545 to amplify the voltage provided to pushing transducer 505, which, in turn, is in communication with function generator 550. Function generator 550 is enabled to excite pushing transducer 505. According to some implementations, function generator 550 is enabled to excite pushing transducer 505 at a frequency of about 20 MHz. According to some implementations, pushing transducer 505 comprises a circular piezoelectric transducer operating at about 20 MHz and having a diameter of about 8.5 mm, an f-number of about 2.35, and transmitting sine-wave bursts of about 400 μs. According to some implementations, pushing transducer 505 comprises a higher frequency ultrasound transducer (i.e. having a frequency above 20 MHz). According to some related implementations, pushing transducer 505, as a higher frequency ultrasound transducer, will also fit into hybrid (OCT imaging/ARF) catheter 510.

According to some implementations, pushing transducer 505 is approximately 2 to 3 mm in diameter. For example, pushing transducer 505 can fit into a 6 French to 9 French gauge catheter.

In summary, herein described are methods and systems for determining mechanical properties of arterial tissues samples by propagating shear waves in arterial tissue samples with ARF, and measuring the shear wave speed and its associated properties with OCT phase maps using an ARF/OCE system, such as the above-described SS-OCT system. The described ARF/OCE methods and systems provide a lower cost and less complex approach to determining the mechanical properties of arterial tissue samples than known US and MRI techniques. In comparison to US and MRI, OCE possesses a higher resolution, which allows for the identification of micron sized morphological tissue structures. Furthermore, in contrast to other elastography methods and systems, such as IVUS, the described ARF/OCE methods and systems can provide non-invasive near-cellular-level imaging for plaque quantification. Understandably, for at least these reasons, the described ARF/OCE methods and systems can be particularly advantageous for the identification of atherosclerotic plaque in arterial tissue samples.

Those skilled in the art will appreciate that in some implementations, the functionality of systems 200 and 500 can be implemented using pre-programmed hardware or firmware elements (e.g., application specific integrated circuits (ASICs), electrically erasable programmable read-only memories (EEPROMs), etc.), or other related components. In other implementations, the functionality of systems 200 and 500 can be achieved using a computing apparatus that has access to a code memory (not shown) which stores computer-readable program code for operation of the computing apparatus. The computer-readable program code could be stored on a computer readable storage medium which is fixed, tangible and readable directly by these components, (e.g., removable diskette, CD-ROM, ROM, fixed disk, USB drive). Furthermore, it is appreciated that the computer-readable program can be stored as a computer program product comprising a computer usable medium. Further, a persistent storage device can comprise the computer readable program code. It is yet further appreciated that the computer-readable program code and/or computer usable medium can comprise a non-transitory computer-readable program code and/or non-transitory computer usable medium. Alternatively, the computer-readable program code could be stored remotely but transmittable to these components via a modem or other interface device connected to a network (including, without limitation, the Internet) over a transmission medium. The transmission medium can be either a non-mobile medium (e.g., optical and/or digital and/or analog communications lines) or a mobile medium (e.g., microwave, infrared, free-space optical or other transmission schemes) or a combination thereof.

Persons skilled in the art will appreciate that there are yet more alternative implementations and modifications possible, and that the above examples are only illustrations of one or more implementations. The scope, therefore, is only to be limited by the claims appended hereto.

What is claimed is:

1. A method comprising:
generating a shear wave in an arterial tissue sample by applying an acoustic impulse thereto;
measuring propagation of the shear wave via an optical coherence elastography apparatus;
determining at least one mechanical property of the arterial tissue sample based on the propagation of the shear wave; and
comparing the at least one mechanical property of the arterial tissue sample to a reference data set to determine whether the arterial tissue sample comprises atherosclerotic plaque.

2. The method of claim 1, wherein the shear wave is generated using an ultrasound transducer.

3. The method of claim 1, wherein the acoustic impulse comprises an acoustic radiation impulse force.

4. The method of claim 1, wherein the optical coherence elastography apparatus comprises an acoustic radiation force-optical coherence elastography.

5. The method of claim 1, wherein the optical elastography apparatus comprises a swept source optical tomography system.

6. The method of claim 1, wherein the at least one mechanical property comprises one or more of a Young's modulus and a shear modulus.

7. A system comprising:
a transducer for generating a shear wave in an arterial tissue sample by applying an acoustic impulse thereto;
an optical coherence elastography apparatus for measuring propagation of the shear wave; and
a computer enabled to
determine at least one mechanical property of the arterial tissue sample based on the propagation of the shear wave, and
compare the at least one mechanical property of the arterial tissue sample to a reference data set to determine whether the arterial tissue sample comprises atherosclerotic plaque.
